(19) United States
(12) Patent Application Publication
(10) Pub. No.: US 2010/0279979 A1
(43) Pub. Date: Nov. 4, 2010

(54) IMMUNOPOTENTIATING AGENT FOR USE IN ANIMALS

(75) Inventors: Nino Sorgente, Los Angeles, CA (US); Philip A. Courier, Jr., Florence, KY (US); Amy J. Miles, Edgewood, KY (US); Arun K. Bahl, Monroe, NC (US)

Correspondence Address: Lisa A. Haile, J.D., Ph.D.;DLA PIPER LLP(US), Suite 1100, 4365 Executive Drive, San Diego, CA, 92121-2133, US

(73) Assignee: IMMUDYNE, INC.

(21) Appl. No.: 12/233,293

(22) Filed: Sep. 18, 2008

Related U.S. Application Data
(63) Continuation of application No. 10/397,047, filed on Mar. 24, 2003, now abandoned.
(60) Provisional application No. 60/443,806, filed on Jan. 29, 2003.

(51) Int. Cl. A61K 31/716; C07H 1/06; A61P 1/00; A61P 3/00

(52) U.S. Cl. 514/54; 536/123.12

(57) ABSTRACT

A simplified method for producing an immunopotentiating agent from cell walls of yeast, other fungi or bacteria is provided and its use as an additive to animal feed to increase resistance to various infections and to potentiate the effect of vaccines is described.

### RELATED APPLICATION

This application is a continuation of co-pending U.S. Ser. No. 10/397,047, filed Mar. 24, 2003, which claims the benefit under 35 U.S.C. §119(e) to U.S. Application Ser. No. 60/443,806, filed Jan. 29, 2003, now abandoned. The disclosures of each of the prior applications are considered part of and are incorporated by reference in the disclosure of this application.

### FIELD OF INVENTION

The present invention relates to a process for the large-scale extraction from fungi or bacterial cell walls of a biologically active carbohydrate, which consists of a beta (1→3) glucan main chain with beta (1→6) glucan side chains and chemically is poly-(1-3)-β-D-glucopyranosyl-(1-6)-β-D-glucopyranose and more simply referred to as beta-1,3/1,6-D-glucan. The invention also relates to the use of beta-1,3/1,6-D-glucans as an animal feed additive in place of antibiotics as growth enhancers, to boost immune systems, combat infections and decrease the bacterial load normally present in animals.

### BACKGROUND OF THE INVENTION

Infectious diseases are the third leading cause of death in the United States, behind heart disease and cancer, and antibiotics are often necessary in treatment of infectious diseases. However, bacteria can develop resistance to an antibiotic upon repeated use so that antibiotics that once were effective to treat infections caused by the bacteria are no longer lethal against the bacteria. Such antibiotic resistance is a serious human health problem and has contributed to the increased cost of treating infectious diseases. Research has linked the use of antibiotics in agriculture to the emergence of antibiotic-resistant strains of disease-causing bacteria. Antibiotics are used in agriculture to treat and prevent diseases in animals and food plants and as feed additives to improve the growth rate of animals.

The most common bacteria found in animals and which are known to cause illness in humans are Salmonella, Campylobacter and Escherichia coli. Although the ill effects of these food-borne pathogens are generally mild, each year several thousand persons contract severe illness and die as a result of exposure to such bacteria. In the United States an estimated 800,000 to 4 million cases of Salmonella infection occur each year, requiring 8000 to 18,000 hospitalizations and resulting in 500 deaths. Similarly, E. coli infections cause 50 to 100 deaths each year in the United States. In addition, of the 2 to 4 million people infected each year in the United States with Campylobacter, 1 in 1000 contract Guillan-Barr syndrome, a disease associated with paralysis.

The first instance of antibiotic-resistant infection in humans in the United States was caused by fluoroquinolone-resistant Campylobacter and was observed in 1996, shortly after fluoroquinolones were approved for use in poultry (The United States General Accounting Office, Report No.: RCED-99-74).

Recently three studies published in the New England Journal of Medicine report that (1) meat sold in grocery stores contains antibiotic resistant Salmonella strains (White et al., N. Engl. J. Med., 345:1147-1154, 2001) and (2) antibiotic resistant strains of Enterococcus faecium from chicken and pork are directly transferred to humans (McDonald et al., N. Engl. J. Med., 345: 1155-1160, 2001 and Sorensen et al., N. Engl. J. Med., 345: 1161-1166, 2001).

The European Union's concern that use of antibiotics in agriculture leads to antibiotic-resistant bacteria that can infect man has resulted in a ban on the use of growth-promoting antibiotics for agricultural purposes in Europe. In the United States, the Center for Disease Control and the Department of Health are also in favor of a ban or a decrease in the use of antibiotics in agriculture. However, industry representatives argue that a ban on use of growth-promoting antibiotics would increase the cost of farming animals, increase the cost of food, and decrease the food supplies.

Some efforts have been made to develop alternatives to the use of growth promoting antibiotics; however, to date there is no satisfactory substitute for antibiotics. It has often been suggested that non-specific immunopotentiating agents would be useful in combating infection by a variety of microorganisms, including bacteria, viruses, fungi, etc. Among the immunopotentiating agents that have been investigated to enhance the activity of the immune system in humans and animals is a polysaccharide, beta-glucan, particularly the beta-glucan derived from the yeast Saccharomyces cerevisiae.

Beta glucans are a family of polysaccharides widely distributed in nature. The beta glucans isolated to date have varied biological activities, such as antifungal, antibacterial (Babineau et al. Randomized phase I/II trial of a macrophage specific immunomodulator (PGG-glucan) in high-risk surgical patients. Ann. Surg. 220:601-609, 1994), and antineoplastic activities (Mansell et al. Clinical Experiences with the use of glucan. In “Immune Modulation and Control of Neoplasia by Adjuvant Therapy. M. A. Chirgos ed., 1978. Raven Press, N.Y., pp. 255-280; Ueno, H. Beta-1,3-D-Glucan, its Immune Effect and its Clinical Use. Japanese Journal Society Terminal Systemic Diseases. 6:151-154, 2000; and (U.S. Pat. No. 4,138,479). These activities appear to be related to a specific structure of beta glucan, namely a beta (1→3) glucan with beta (1→6) side chains at varying positions and in varying amounts, and which have the chemical designation of poly-(1-3)-β-D-glucopyranosyl-(1-6)-β-D-glucopyranose. The distribution and quantity of beta (1→6) side chains appears to influence intensity of the activity. A number of these modified beta glucans have been purified to varying degree and from various sources.

Many therapeutic activities have been attributed to these modified beta glucans and an abundance of claims have been made. It is difficult to assess the validity of many of these claims since investigators have used preparations of differing degrees of purity and obtained by different methodologies, and some investigators have reported no effects or effects opposite to those reported by others.

There have been a number of reports regarding the purification and uses of beta glucan from yeast, including its use in cosmetics (U.S. Pat. No. 5,223,491), to enhance resistance to diseases in aquatic animals (U.S. Pat. No. 5,401,727), and as a nutritional supplement for man and animals (U.S. Pat. No. 5,576,015). The methods described in these patents are time consuming and the procedures described yield small quantities. Whether any of these methods can produce an active beta glucan when obtained by large-scale manufacturing is not known.

For example, a number of procedures have been described for preparing insoluble beta glucan. Most of these procedures are based on alkali extraction of yeast, bacteria, fungi, or the cell walls of these organisms. followed by an acid extraction and subsequent extractions with various organic solvents. These procedures usually yield small quantities of glucan, very often without any regard to biological activity. For example, U.S. Pat. No. 5,401,727 describes purification of beta glucan from 500 grams of Saccharomyces cerevisiae (with no yield given); U.S. Pat. No. 5,223,491 describes two procedures using 500 and 200 grams of Saccharomyces cerevisiae yielding 50 and 20 grams of purified beta glucan, respectively; U.S. Pat. No. 6,242,594 describes preparation of a glucan using 400 grams of Saccharomyces cerevisiae as the starting material. The time required for preparing such amounts glucan varies from a minimum of 8 hours to a few days.

Therefore, there is a need in the art for new and better methods for preparation of large quantities of active beta glucan needed for use in agriculture, for example large, commercial-scale production of beta glucans that are active as an immunoactivator in the field as well as in the laboratory.

### SUMMARY OF THE INVENTION

The invention overcomes these and other problems in the art by providing reproducible, efficient and rapid procedure for the large-scale manufacture of an active, immunomodulating beta glucan from organisms selected from fungi and bacteria, especially from the cell walls of such organisms. When manufactured according to the invention methods for large scale production, the beta glucan from Saccharomyces cereviciae cell walls is a potent activator of the immune system and effective in combating infections in the laboratory as well as in the field. More generally the present invention is based on the discovery of a method for large-scale manufacture of beta (1→6) branched beta (1→3) glucan that is active as an immunomodulator and is sufficiently cost-effective that the beta glucan can be used as an additive in feeds for farmed animals, for example to eliminate antibiotics from the diet or decrease their use, to increase resistance to infections and to increase vaccine effectiveness.

Accordingly, in one embodiment, the invention provides methods for large-scale production of beta (1→3)/(1→6)-D-glucan wherein a mixture comprising at least 1200 pounds by dry weight of cell walls of an organism selected from fungi and bacteria and a 0.5 N to 5.0 N alkaline solution of an alkali-metal or alkali-earth metal hydroxide is heated to a temperature of about 45° C. to about 80° C. with stirring for about 30 minutes. The mixture is then pressurized to about 5 psi to about 30 psi at a temperature in the range from about 100° C. to about 121° C. for about 15 min to about 120 min. After the pressurization treatment, solids are separated from the mixture and subjected to an acid solution in a ratio of about 1:1 to about 1:10 solids to acid solution while being heated to a temperature of from about 50° C. to about 100° C. for 15 minutes to about 2 hours. Solids separated from the acid treatment step will comprise at least 75% by dry weight of beta (1→3)/(1→6)-D-glucan.

In another embodiment, the invention provides an animal feed comprising beta (1→3)/(1→6)-D-glucan prepared by the invention methods in an amount effective for enhancing growth of an animal fed on the feed at least during the growth period of the animal.

In yet another embodiment, the invention provides methods for enhancing growth of poultry by adding an effective amount of beta (1→3)/(1→6)-D-glucan produced from cells of Saccharomyces cerevisiae to feed of growing poultry, thereby enhancing the growth of the poultry.

### DETAILED DESCRIPTION OF THE INVENTION

In one embodiment, the invention provides methods for large-scale production of beta (1→3)/(1→6)-D-glucan. Large quantities of bacterial or fungal cells or cell walls, usually 1200 pounds to about 1600 pounds dry weight of cell walls, is the starting material. This starting material is mixed with a 0.5 N to 5.0 N alkaline solution of an alkali-metal or an alkali-earth metal hydroxide, such as sodium hydroxide or potassium hydroxide, and heated to a temperature of about 45° C. to about 80° C. with stirring for about 30 minutes. The mixture is then pressurized to about 5 psi to about 30 psi at a temperature in the range from about 100° C. to about 121° C. for about 15 min to about 120 min. Then the mixture is cooled and solids are separated from the mixture, for example using multiple steps of washing and centrifugation using an industrial scale centrifuge. Separated solids are subjected to an acid treatment using a ratio of about 1:1 to about 1:10 solids to acid solution while being heated to a temperature of from about 50° C. to about 100° C. for 15 minutes to about 2 hours. Solids separated from the acid treatment step will comprise at least 75% by dry weight of beta (1→3)/(1→6)-D-glucan.

The preferred source of cell walls for use in the invention large-scale production methods is the yeast Saccharomyces cerevisiae, from whose cell walls about 85% by dry weight of the beta (1→3)/(1→6)-D-glucan can be obtained.

The method optionally further comprises sterilizing the dry solids obtained in this manner using a sterilization technique that is non-toxic to animals, for example irradiation.

In addition to yeast, such as Saccharomyces cerevisiae, the invention methods can be used to prepare beta glucans from other fungi, such as, for example, the mushroom Blazei agaricus, as well as from Blazei agaricus and various Yunzhi.

An “effective amount” of beta glucan for use in promoting healthy growth in an animal is an amount sufficient to promote at least one of the following: inhibition of bacterial load in the animal; prevention or decrease the incidence of necrotic enteritis in poultry; stimulation of the immune response in the animal; enhancement of the effectiveness of antibiotics and vaccines administered to the animal in feed or otherwise; increased growth rate per amount of feed administered, and the like. Those of skill in the art will consider such factors as the animal's age, level of activity, hormone balance, and general health in determining the effective amount, which is tailored to the animal, for example by beginning with a low dosage and titrating the dosage to determine the effective amount.

Animals that can benefit from ingesting the invention animal feed and from treatment using feeds containing an effective amount of beta (1→3)/(1→6)-D-glucan are all types of fanned poultry, including, for example, chickens, ducks, geese, turkeys, quail, game hens, and the like. Other farmed animals that can benefit from feed containing an effective amount of beta (1→3)/(1→6)-D-glucan as described herein include, for example, beef and dairy cattle, pigs, goats, salmonids and the like

### A. Method for the Large-Scale Preparation of Beta (1→3)/(1→6)-D-Glucan.

Dry yeast or other fungi or dry yeast cell walls are mixed with NaOH in the range of 0.5 to 5.0 N, and preferably 1.5 N NaOH. The mixture is then heated to about 45° C. to 80° C., and preferably about 60° C., with stirring and is kept at this temperature for about 30 minutes with stirring. The temperature is then increased to a temperature in the range from about 100° C. to about 121° C., and the mixture is placed under a pressure between about 5 psi and about 30 psi, more preferably at about 121° C. and about 15 psi of pressure, for about 15 min to about 120 min. The mixture is then allowed to cool and the liquid is separated from the solids. The solids are washed 1 to about 3 times with 1 to about 10 volumes of water.

The washed solids are separated from the liquid and an acid, such as hydrochloric or acetic acid is added. For example, about 3% acetic acid can be added in a ratio of about 1:1 to about 1:10 solids to acid. The mixture is then heated to between about 50° C. and 100° C. for 15 minutes to about 2 hours. More preferably the mixture is heated to 85° C. for about 45 minutes. The hot mixture is allowed to cool and the solids, which are comprised of approximately 80% beta (1→3)/(1→6)-D-glucan, are separated from the liquid and again washed 1 to about 3 times with 1 to about 10 volumes of water.

The solids are separated from the liquid and dried in ambient temperature or warm air, warmed in an oven, or spray dried, with spray drying being preferred. The dried purified beta (1→3)/(1→6)-D-glucan can then be sterilized, for example by irradiation. When prepared as above described, the spray dried beta (1→3)/(1→6)-D-glucan contains about 85% to about 98% beta (1→3) and the remainder beta (1→6) bonds, as analyzed by Nuclear Magnetic resonance.

Biologically, beta (1→3)/(1→6)-D-glucan activates the alternative complement pathway and stimulates the release of nitric oxide from macrophages in vitro.

### B. Use of Beta (1→3)/(1→6)-D-Glucan in Feed for Poultry.

The successful farming of animals, and thus the low cost of meat, depends on use of antibiotics added to animal feed and use of antibiotics to treat diseases as they occur during the growth of the animal. However, excessive use of antibiotics can be quite harmful to humans because its use generates resistant strains of bacteria that can infect humans. To determine whether beta (1→3)/(1→6)-D-glucan can substitute for antibiotics, beta (1→3)/(1→6)-D-glucan was added to chicken feed at concentrations in the range between 5 grams and about 500 grams per ton of feed, for example between 20 and 40 grams per ton. Chickens were fed this diet until market age. Weight, feed conversion rate, mortality and condemnation rate were recorded and compared to those of chickens fed regular diets containing antibiotics as well as diet containing probiotics, or diets containing no growth promoting additives. C. The use of beta (1→3)/(1→6)-D-glucan to prevent necrotic enteritis in chickens.

Necrotic enteritis is an enterotoxemic disease in chickens caused by Clostridium perfringens types A and C. This disease is characterized by sudden onset of diarrhea, explosive mortality, and confluent mucosal necrosis of the small intestine. The condition causes profound depression and rapid death, with mortality rates of more than 1% a day. Clostridium perfringens is considered to be widespread in the environment. Because Clostridia can produce spores, and these spores are very resistant to environmental conditions, infections are common. Spores remain in a house in which an infected flock is kept. Spores may also occur in feed. It is assumed that the heat produced in pelleting chicken feed will not destroy the spores. Consequently, risk of flocks becoming infected is considered high.

To prevent or decrease the incidence of necrotic enteritis beta (1→3)/(1→6)-D-glucan is added to chicken feed at a concentration between about 5 grams and about 500 grams per ton of feed, for example, between about 10 grams and about 100 grams or between about 20 grams and about 40 grams per ton. Chickens are generally fed this diet until market age. Accordingly, in another embodiment, the invention provides animal feed comprising beta (1→3)/(1→6)-D-glucan prepared by the invention large-scale method in an amount effective for enhancing growth of an animal consuming the feed at least during the growth period of the animal. An effective amount of the beta (1→3)/(1→6)-D-glucan for enhancing growth can be, for example, in the range from about 5 grams to about 500 grams per ton of the feed, in the range from about 10 grams to about 100 grams per ton of the feed, or in the range from about 20 grams to about 40 grams per ton of the feed. The invention animal feed will additionally contain a staple food as is known in the art selected for the animal for which it is intended. For example, for chickens, the invention feed can additionally comprise any of the constituents considered in the art as suitable for chicken feed.

In yet another embodiment, the invention provides methods for enhancing growth of poultry by adding an effective amount of beta (1→3)/(1→6)-D-glucan, as described herein, produced from cells of Saccharomyces cerevisiae to poultry feed of growing poultry at least during the growth period of the poultry, thereby enhancing the growth of the poultry. The term “enhancing growth” as used herein is intended to include such specific advantages as treating, i.e., inhibiting, preventing, or curing, necrotic enteritis in the poultry, reducing the bacterial load in the poultry, and enhancing the immune system of the poultry.

In still another embodiment, the invention provides an animal feed additive comprising beta (1→3)/(1→6)-D-glucan, wherein the animal feed additive is produced by the invention methods. Preferably the feed additive beta (1→3)/(1→6)-D-glucan produced from cells of Saccharomyces cerevisiae.

The invention is further illustrated by the following non-limiting examples.

### Example 1

### Large Scale Separation of Beta (1→3)/(1→6)-D-Glucan from Yeast Cell Walls

With stirring, 1600 lb of yeast cell walls were mixed with 1300 gallons of 1.5 N NaOH. The mixture was heated to 60° C. with stirring and kept at 60° C. with stirring for 30 min. The temperature was then increased to 121° C. and the vessel containing the mixture was pressurized to 15 psi with stirring for 15 to 45 minutes. The mixture was then cooled to safe handling temperature and adjusted to 17% to 27% solids. The mixture was separated using a Westfalia separator, model SC-35 (Westfalia A. G., Oelde, Germany). The separated solids were washed by dilution with water to about 26% solids using a ZA4 centrifugal mixer (Westfalia A. G., Oelde, Germany) and again separated on the Westfalia separator. The water washes are done 1-2 times and preferably 2 times. The solids were combined with approximately 100 gallons of 3% acetic acid and transferred to a tank containing 800 gallons of 3% acetic acid at 85° C. The mixture was heated to 85° C. for 45 minutes. The mixture was again cooled to safe handling temperature and adjusted to 17% to 27% solids. The mixture was once again separated using a Westfalia separator, model SC-35, and the separated solids were washed by dilution with water to about 26% solids and again separated on the Westfalia separator.

The yield of beta glucan is 110 kg and the average time needed for the above-described preparation was about 25 hours. Nuclear Magnetic Resonance analysis of a typical lot prepared by this method shows that the beta glucan contains 80% carbohydrate and specifically beta (1→3)/(1→6)-D-glucan with a beta (1→3) to beta (1→6) ratio of 10.

### Example 2

The beta (1→3)/(1→6)-D-glucan prepared using the methods disclosed herein yields a product that is biologically active and the biological activity is reproducible from lot to lot. To determine biological activity we measured the activation of the alternative complement pathway. The assays were carried out by a commercial laboratory (The Complement Laboratory, National Jewish Medical and Research Center, Denver, Colo., USA). The assay consists of mixing 1 part of a suspension of beta glucan with 9 parts of human serum. After 30 minutes of incubation at 37° C., the mixture is centrifuged and analyzed quantitatively for Bb, a protein fragment released upon activation of the complement protein Factor B.

TABLE 1

| | Activity |
|---|---|
| Lot No | (μg Bb Released) |
| IM620 | 40.5 |
| IM 301 | 46.0 |
| IM 015 | 49.0 |
| IM 104 | 47.5 |
| IM 119 | 47.0 |
| IM 204 | 57.8 |
| IM 310 | 40.6 |
| IM 331 | 56.0 |
| IM 426 | 45.7 |
| IM 503a | 53.5 |

The average activity of the 10 lots of Immustim® (IM) in Table 1 is 48.36 μg Bb released/mg of Immustim®. The positive controlled used in the assays was Zymosan, which is an alcoholic extract of the yeast Saccharomyces cerevisiae containing between 30 and 40% beta-1,3/1,6-D-glucan. The average activity of Zymosan was only 9.6 μg Bb released/mg, even though Zymosan contains 40-50% of the beta-1,3/1,6-D-glucan of Immustim®, suggesting that beta-1,3/1,6-D-glucan in yeast cell walls is not available to activate complement.

### Example 3

### Field Trials Showing the Effect of Immustim® on the Growth of Chickens

These studies were performed on 47 farms with a total of 1,402,015 chickens. Chickens were fed either the standard diet containing the antibiotics virginiamycin (20 g/ton) and salinomycin (60 g/ton) or they were fed a diet containing the coccidiostat Amprol (250 ppm) and beta (1→3)/(1→6)-D-glucan, Immustim®, at 40 grams per ton for the first 2 weeks and 20 grams per ton for the following 4 weeks. At the end of the six weeks, performance was assessed using the following criteria: mortality, weight, feed conversion, condemnation rate. The results of this experiment summarized in Table 2 below show comparable growth parameters in chickens fed on the two feed regimens, indicating that it is feasible to farm chickens without antibiotics.

TABLE 2
Effect of beta (1→3)/(1→6)-D-glucan on growth of Chickens

| | Antibiotics + Salinomycin¹ | Immustim ® plus |
|---|---|---|
| Criteria | (30 Farms) | Amprol¹ (17 Farms) |
| Mortality (%) | 4.40 | 4.70 |
| Age (days) | 46.5 | 46.9 |
| Weight (lb) | 5.15 | 5.13 |
| Feed Conversion² | 2.00 | 2.01 |
| Condemnation % | 1.32 | 1.24 |

¹Salinomycin (Alpharma, Fort Lee, N.J., USA and Amprol (Merial Ltd., Athens, GA, USA) are coccidiostats, agents for the control of coccidia intracellular parasites.
²Feed conversion is based on net sellable meat basis, after shrink, DOC, whole bird and parts condemnation.

### Example 4

### Reduction of Bacterial Load by Beta (1→3)/(1→6)-D-Glucan

To determine the effectiveness of beta (1→3)/(1→6)-D-glucan in decreasing bacterial load in poultry, turkeys were fed a control diet containing probiotics or a diet containing 40 grams of (1→3)/(1→6)-D-glucan, Immustim®, per ton of feed for the first 6 weeks followed by 20 grams per ton. Early morning cecal droppings were collected and the level of salmonella and campylobacter determined. The results of this experiment are summarized in Table 3 below:

TABLE 3
Effect of beta (1→3)/(1→6)-D-glucan on Bacterial Load in Turkeys

| | Probiotics | Immustim ® |
|---|---|---|
| Salmonella | 7/24 | 4/24 |
| Campylobacter | 21/24 | 13/24 |

The data in Table 3 indicates that the bacterial load is decreased by about 150% in turkeys treated with beta (1→3)/(1→6)-D-glucan as compared to the bacterial load in turkeys treated with probiotics.

### Example 5

### Effect of Beta (1→3)/(1→6)-D-Glucan on Necrotic Enteritis in Poultry

Necrotic enteritis, a disease that affects the gut of chickens, results in high mortality rates when it manifests itself clinically; sub-clinically the disease results in decreased growth Necrotic enteritis is a major problem in growing chickens, especially in the absence of growth promoting antibiotics. To test the effect of beta glucan feed supplement in a field trial, chickens were fed either the standard feed which contained the antibiotic flavomycin (2 g/ton) and the ionophore biocox (60 gm/ton), or they were fed a diet containing flavomycin (2 g/ton) plus beta (1→3)/(1→6)-D-glucan at 40 grams per ton for the first 2 weeks and 20 grams per ton for the following 4 weeks. The results of these studies summarized in Table 4 below show that beta (1→3)/(1→6)-D-glucan was effective in preventing necrotic enteritis.

TABLE 4
Effect of beta (1→3)/(1→6)-D-glucan on Necrotic Enteritis
in Chickens Comparison between Antibiotic and
Antibiotic + Immustim ®

| | | Necrotic | |
|---|---|---|---|
| House | Treatment | enteritis | Treatment |
| 4 | Flavomycin¹ + Salinomycin | +++ | Penicillin 3 days |
| 5 | Flavomycin + Salinomycin | +++ | Penicillin 3 days |
| 6 | Flavomycin + Immustim ® | − | None |
| 7 | Flavomycin + Immustim ® | − | None |

¹Flavomycin (Hoescht Roussel GmbH, Germany)

### Example 6

### Effect of Beta (1→3)/(1→6)-D-Glucan on Necrotic Enteritis in Chickens

In this trial, chickens were administered Cocci Vac vaccine (Schering-Plough Animal Health Corp., Kenilworth, N.J., USA), a vaccine against coccidia (an intracellular parasite), in an aerosol form or fed Cocci Vac (Cocci Vac I, a biological vaccine distributed in unit doses; one animal gets one dose) and a diet supplemented with beta (1→3)/(1→6)-D-glucan at 40 grams per ton for the first 2 weeks and 20 grams per ton for the following 4 weeks. The results of this comparison study are summarized below in Table 5.

TABLE 5
Effect of beta (1→3)/(1→6)-D-glucan on Necrotic Enteritis
in Chickens Comparison between Antibiotic-free and
Immustim ®

| House | Treatment | Necrotic enteritis | Treatment |
|---|---|---|---|
| 1 | Cocci Vac¹ | +++ Multiple outbreaks | Penicillin each time |
| 2 | Cocci Vac | +++ Multiple outbreaks | Penicillin each time |
| 3 | Cocci Vac | +++ Multiple outbreaks | Penicillin each time |
| 4 | Cocci Vac + | + Mild outbreak | None |
| | Immustim ® | | |
| 5 | Cocci Vac + | − | None |
| | Immustim ® | | |
| 6 | Cocci Vac + | − | None |
| | Immustim ® | | |

¹Cocci Vac is a vaccine to protect the chickens against the coccidian parasites.

The data above in Table 5 indicates that beta (1→3)/(1→6)-D-glucan prepared according to the methods disclosed herein is very effective in preventing necrotic enteritis both in the presence and absence of antibiotics in the feed.

### Example 7

The beta (1→3)/(1→6)-D-glucan prepared according to the methods disclosed herein is also effective in protecting aquatic animals from infections. To determine the effectiveness of beta(1→3)/(1→6)-D-glucan in preventing infections in aquatic animals, survival of shrimps (L. vannamei) infected with the White Spot Syndrome Virus (WSSV) and fed diets containing 0, 50, 100, or 500 gram/ton of beta (1→3)/(1→6)-D-glucan prepared as described herein was studied. The results of this study are shown in Table 6 below.

TABLE 6
Survival of Shrimps infected with WSSV and fed diets
containing different amounts of beta (1→3)/(1→6)-D-glucan

| Treatment | |
|---|---|
| (g Beta- | |
| glucan/ton feed) | Survival (%) |
| 0 | 23.1 |
| 50 | 45.5 |
| 100 | 70.6 |
| 500 | 27.3 |

The data in Table 6 clearly shows that the beta (1→3)/(1→6)-D-glucan prepared as described herein is effective in substantially increasing survival in shrimps infected with WSSV. In addition, it shows that it is necessary that dosage be evaluated, because at high doses the effectiveness is lost, which is likely the result of receptor down-regulation.

Although the invention has been described with reference to the presently preferred embodiment, it should be understood that various modifications can be made without departing from the spirit of the invention. Accordingly, the invention is limited only by the following claims.

What is claimed is:

1. A method for large-scale production of beta (1→3)/(1→6)-D-glucan, said method comprising:
a) heating a mixture comprising at least 1200 pounds by dry weight of cell walls of an organism selected from fungi and bacteria and a 0.5 N to 5.0 N alkaline solution of an alkali-metal or an alkali-earth metal hydroxide to a temperature of about 45° C. to about 80° C. with stirring for about 30 minutes;
b) pressurizing the mixture to a pressure from about 5 psi to about 30 psi at a temperature in the range from about 100° C. to about 121° C. for about 15 min to about 120 min;
c) subjecting solids separated from the mixture of b) to an acid solution in a ratio of about 1:1 to about 1:10 solids to acid solution while heating to a temperature of about 85° C. for about 45 minutes; and
d) separating solids obtained from c); wherein the solids comprise at least 75% by dry weight of beta (1→3)/(1→6)-D-glucan.

2. The method of claim 1, wherein the temperature in a) is about 60° C. and the alkaline solution is a solution of Na.

3. The method of claim 1, wherein the cell walls are obtained from Saccharomyces cerevisiae and wherein about 85% by dry weight of the beta (1→3)/(1→6)-D-glucan is separated in d).

4. The method of claim 1, wherein the fungi is one or more yeast.

5. The method of claim 1, wherein the cell walls are obtained from bacteria.

6. The method of claim 1, wherein the acid is selected from hydrochloric and acetic acid.

7. The method of claim 6, wherein the acid is 3% acetic acid.

8. The method of claim 1, wherein in step b) the temperature is about 121° C. and the pressure is about 15 psi.

9. The method of claim 1, wherein in c) the temperature is 85° C. for about 15 minutes.

10. The method of claim 1, further comprising:
e) sterilizing the dry solids.

11. The method of claim 10, wherein the dry solids are sterilized by irradiation.

12. The method of claim 1, wherein the separating is by centrifugation.

13. An animal feed comprising beta (1→3)/(1→6)-D-glucan prepared by the method of claim 1 in an amount effective for enhancing growth of an animal fed on the feed at least during the growth period of the animal.

14. The animal feed of claim 13, wherein the effective amount is a concentration of the beta (1→3)/(1→6)-D-glucan in the range from about 5 grams to about 500 grams per ton of the feed.

15. The animal feed of claim 14, wherein the effective amount is a concentration of the beta (1→3)/(1→6)-D-glucan in the range from about 10 grams to about 100 grams per ton of the feed.

16. The animal feed of claim 15, wherein the effective amount is a concentration of the beta (1→3)/(1→6)-D-glucan in the range from about 20 grams to about 40 grams per ton of the feed.

17. The animal feed of claim 13, wherein the animal feed further comprises a poultry feed staple.

18. The animal feed of claim 17, wherein the poultry feed staple is selected from chicken feed and turkey feed.

19. The animal feed of claim 13, wherein the animal feed further comprises a feed staple suitable for feeding beef cattle.

20. A method for enhancing growth of poultry, said method comprising adding an effective amount of beta (1→3)/(1→6)-D-glucan produced from cells of Saccharomyces cerevisiae to poultry feed of growing poultry at least during the growth period of the poultry, thereby enhancing the growth of the poultry.

21. The method of claim 20, wherein the effective amount is between 5 and 500 grams per ton of feed.

22. The method of claim 20, wherein the effective amount is 40 grams per ton during the first two weeks of growth and 20 grams for the rest of the life of the poultry.

23. The method of claim 20, wherein enhancing growth includes treating necrotic enteritis in the poultry.

24. The method of claim 23, wherein the effective amount is between about 5 grams and about 500 grams per ton of feed.

25. The method of claim 7 wherein the effective amount is about 40 grams per ton during the first two weeks of growth and about 20 grams for the rest of the life of the poultry.

26. The method of claim 25, wherein bacterial load in the poultry is decreased.

27. The method of claim 25, wherein immune system of poultry is enhanced.

28. An animal feed additive comprising beta (1→3)/(1→6)-D-glucan produced by the method of claim 1.

29. The feed additive of claim 28, wherein the feed additive is added to shrimp feed.

30. The feed additive of claim 29, wherein the effective amount is from 25 to 300 grams per ton of the feed.

31. The feed additive of claim 30, wherein the effective amount is about 100 grams/ton of the feed.

32. An animal feed additive, wherein the beta (1→3)/(1→6)-D-glucan is produced from cells of Saccharomyces cerevisiae.
